(19) United States
(12) Patent Application Publication
(10) Pub. No.: US 2017/0105862 A1
(43) Pub. Date: Apr. 20, 2017

(54) Leg Lift Walking Aid

(71) Applicant: Sandra V. Griggs, Panama City, FL (US)

(72) Inventor: Sandra V. Griggs, Panama City, FL (US)

(21) Appl. No.: 14/887,668

(22) Filed: Oct. 20, 2015

(51) Int. Cl. A61F 5/01; A61H 3/00

(52) U.S. Cl. CPC A61F 5/0106; A61F 5/0113; A61H 3/00; A61H 2003/007

(57) ABSTRACT

A leg lift walking aid for assisting a person with limit leg functionality is provided. The leg lift walking aid comprises a waist band suitable for wearing around a use and a foot plate capable of being placed under a foot of the user. A pair of straps between the waist band and foot plate are provided wherein each strap comprises at least one flexible portion and at least one non-flexible portion. A central band, suitable for wearing on a leg below a knee of a user comprises at least one guide wherein the guide receives at least one strap and preferably slidably.

### BACKGROUND

The present invention is related a device for assisting those with limited use of a leg to walk in a more normal fashion. More specifically, the present invention allows for an assist in leg lift thereby providing a near normal walking gait.

The result of many diseases is the loss of functionality of a limb and, for the purposes of this invention, the loss of functionality of a leg. With some diseases the loss is reversible, typically through therapy, and with some diseases the loss is irreversible. For those with reversible conditions it is important to the recovery process to provide mobility while the condition is reversed. Increased mobility increases the rate of recovery physically and, possibly more importantly, improves the quality of life of the person during the recovery process. If the condition is not reversible, or is marginally reversible, it is important to provide a modified form of walking thereby allowing unaffected muscles to function in alternate ways to increase mobility thereby improving the quality of life.

When leg functionality is compromised the person has limited ability to lift the leg in a manner that allows the leg to pivot at the hip and bend at the knee. Therefore, as they are attempting to ambulate the weak leg is an impediment. It is therefore important to provide an ability to lift the leg away from the ground as the person ambulates. Yet another problem associated with the loss of leg functionality is a syndrome referred to as “toe-drop”. With toe-drop, as the leg is lifting the lack of muscle control allows the foot to pivot downward at the ankle such that the toes point downward. This is detrimental for several reasons. Toe-drop requires the leg to be lifter even higher to allow the toes to clear an obstacle or to insure the foot does not drag. Also, as the leg is lowered the foot may be in a awkward position which further complicates the ability to ambulate and may cause additional harm as the foot may not be in a natural position when reengaging with the ground.

There has been significant effort in providing an assist for those with loss of leg function. Most of the methods taught in the art either function autonomously, to move the leg in a desired manner mechanically, or involve the use of another limb, such as the arm, to assist in the lifting motion. Though helpful, these methods do not typically assist with foot drop.

In spite of the ongoing effort those of skill in the art still do not have a suitable leg lift walking aid. The art specifically lacks a leg lift walking aid which also corrects for foot drop and which enhances the rehabilitation process by allowing for a more natural gait.

### SUMMARY OF THE INVENTION

It is an object of the invention to provide a leg lift walking device.

It is an object of the invention to provide a device which allows a person with limited use of a leg to walk with a more natural gait by facilitating rotation at the hip and bending at the knee while also counteracting toe drop.

These and other advantages, as will be realized, are provided in a leg lift walking aid. The leg lift walking aid comprises a waist band suitable for wearing around a user; a foot plate capable of being placed under a foot of the user; a pair of straps between the waist band and the foot plate wherein each strap comprises at least one flexible portion and at least one non-flexible portion; and a central band suitable for wearing on a leg, below a knee, of a user wherein the central band comprises a pair of guides wherein each guide receives one strap.

Yet another embodiment is provided in a method of walking with a first leg of limited functionality comprising: wearing a leg lift walking aid wherein the leg lift walking aid comprises: a waist band suitable for wearing around a user above a hip joint; a foot plate capable of being placed under a first foot of the first leg of the user; a pair of straps between the waist band and the foot plate wherein each strap comprises at least one flexible portion and at least one non-flexible portion; and a central band suitable for wearing on the first leg below a knee of the first leg of the user wherein the central band comprises a pair of guides wherein each guide receives one strap; standing upright with the first foot and a second foot on a surface; lifting hips away from the surface wherein the first leg is persuaded to rotate at the hip joint and bend at the knee; ambulating forward; and returning the first foot to the surface.

### FIGURES

FIG. 1 is a schematic view of an embodiment of the invention.

FIG. 2 is a schematic view of an embodiment of the invention.

FIG. 3 is a schematic view of an embodiment of the invention.

### DESCRIPTION

The instant invention is specific to a leg lift walking aid. More specifically, the present invention is specific to a leg lift walking aid capable of simultaneously counteracting foot drop. Even more specifically, the present invention assist the patient in rehabilitation thereby allowing alternate muscle assignment to counteract the loss of functionality.

The invention will be described with reference to the various figures forming an integral non-limiting component of the disclosure. Throughout the disclosure similar elements will be numbered accordingly.

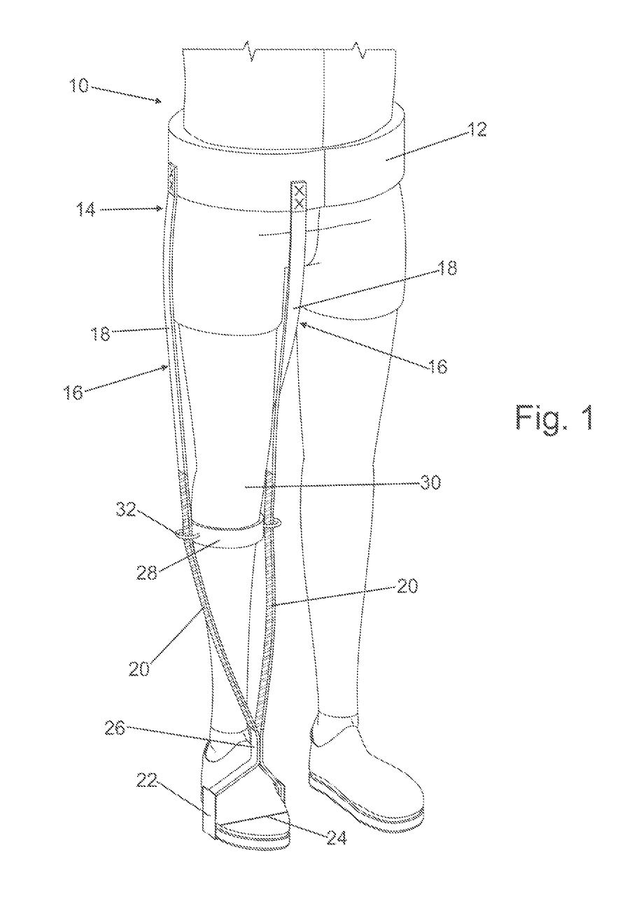

An embodiment of the invention will be described with reference to FIG. 1 wherein a leg lift walking aid is illustrated in perspective schematic view as worn. In FIG. 1, the user, 10, is illustrated in partial schematic view with the leg lift walking aid on the right leg even though it would be understood that the leg lift walking aid could be worn on the left leg. The leg lift walking aid comprises a waist band, 12, circumnavigating the waste just above the hip joint, 14, so as to not impede rotation at the hip joint. It is preferable that the lowest extent of the waist band be at least 25.4 mm (1 inch) above the hip joint to no more than about 30.4 cm (12 inches) above the hip joint. If the lowest extent of the waist band is less than about 25 mm above the hip joint rotation at the hip joint is impeded or the waist band will cause discomfort when the leg is lifted and the leg rotates at the hip joint in concert with the lift. If the waist band is more than 30.4 cm above the waist band the functionality decreases as will be realized further herein. It is preferable that the waist band be sufficiently tight as to remain in place when downward pressure, such as the weight of the leg, is applied. If the waist band slides downward significantly during leg lift the functionality of the leg lift walking aid is compromised. It would be apparent that the waist band can not be so tight as to cause discomfort. It is therefore preferable that the waist band be on the tissue covering the hip bone, since this provides a more comfortable fit and above the hip bone the waist band may cause discomfort if not undergirded by bone structure. The width of the waist band is not particularly limited yet it would be understood from the description that the width must be sufficient to provide adequate support without discomfort. A larger width provides more support yet the comfort may be compromised. If the width is to small the support is compromised. A width of about 7.62 cm (3 inches) to about 30.4 cm (12 inches) has proven to be optimal.

The waist band material is preferably a clothing material as these are widely available and easily configured. Though not required, clothing material with some degree of elasticity is preferred for comfort. In one embodiment an anti-skid material is incorporated into the inner lining of the waist band to discourage sliding downward and to minimize the tautness of the waist band required for stability. A particularly preferred inner lining includes an anti-slip material such as a rubber coated fiber or fabric, preferably, in a screen pattern to minimize weight and to allow air to circulate there through. Grip Liner, Catalog Item #74698, or Grip Placemat, Catalog Item #74710, both available from Camper World are exemplary for demonstration of the invention.

Attached to the waist band and extending downward is at least one, and preferably two straps, 16, and preferably the straps are interchangeable. Each strap comprises at least one fabric which does not readily stretch and at least one portion which does stretch. For the purposes of illustration, the straps in FIG. 1 comprise an upper fabric section, 18, which does not stretch during use and a lower portion, 20, which does stretch during use. The degree of stretching will be described elsewhere herein. The straps extend between the waist band and a foot portion comprising a foot plate, 22, which is situated under the foot of the user, and preferable just behind the toe joint, 24, and preferably between the ankle and toe joint. The straps preferably meet at a transition, 26, which is illustrated in FIG. 1 as a point of joining of the two straps. For the purposes of discussion the strap will be described as an upper strap, which is that portion above the transition, and a lower strap, which is that portion below the transition.

A central band, 28, is attached to the user just below the knee, 30. The central band circumnavigates the leg and preferably does not slide on the leg during use. The central band comprises at least one guide, 32, within which the strap is retained preferably slidably. The central band maintains the strap in a position which is effectively aligned with the leg from the waist band to just below the knee and then the strap is effectively aligned from just below the knee to the foot plate. By effectively aligning the strap with the leg the persuasion of the flexible portion is aligned with the leg and therefore as the leg is bent the persuasion of the flexible portion is along a hypotenuse formed by the triangle having an apex at the knee and one portion of the triangle being the leg segment between the knee and guide and the other portion of the triangle being the upper leg to the attachment point of the strap to the waist band which approximates the hip joint. Without the guide the strap would be along a hypotenuse of the triangle formed by the upper leg and lower leg meeting at the knee, as the apex, which is non-operative. The central strap is preferably about 7.62 cm (3 inches) below the knee but preferably no more than about 15.24 cm (6 inches) below the knee. If the central strap is closer than about 7.62 cm it is difficult to achieve adequate persuasion for the upper leg to rotate at the hip and for the leg to bend at the knee as the leg is lifted. If the central strap is more than about 15.24 cm below the knee the effort required to straighten the leg after bending is excessive and inoperative.

The central band material is preferably a clothing material as these are widely available and easily configured. Though not required, clothing material with some degree of elasticity is preferred for comfort. In one embodiment an anti-skid material is incorporated into the inner lining of the waist band to discourage sliding downward and to minimize the tautness of the waist band required for stability. A particularly preferred inner lining includes an anti-slip material such as a rubber coated fiber or fabric, preferably, in a screen pattern to minimize weight and to allow air to circulate there through. Grip Liner, Catalog Item #74698, or Grip Placemat, Catalog Item #74710, both available from Camper World are exemplary for demonstration of the invention.

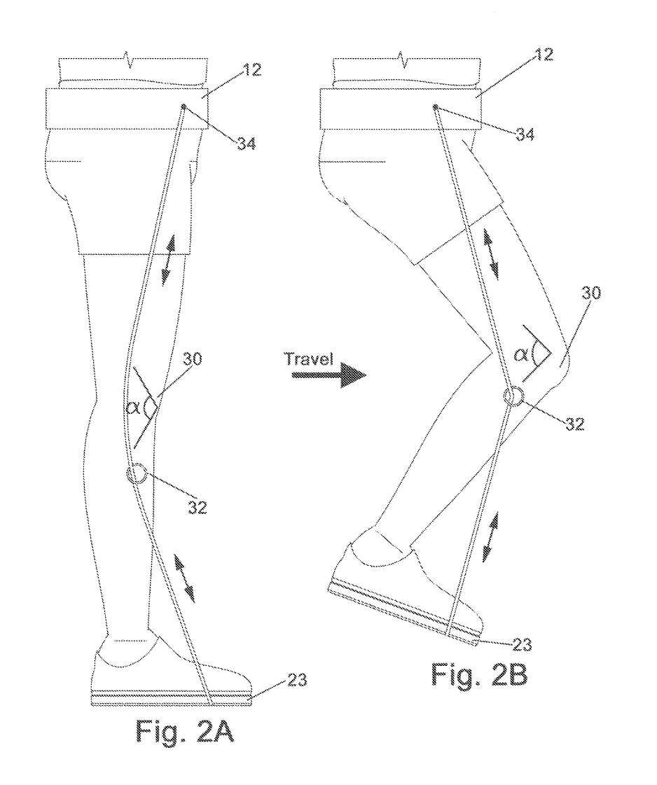

The operation of the leg lift walking device will be described with reference to FIG. 2 wherein a schematic representation of a leg extended, left, and bent, right, is provided. In FIG. 2 the knee 30, forms an apex of a triangle with an angle of a. As would be realized the angle α is larger with a straight leg than with a bent leg. At relatively straight, left view, the strap is extended and those portions of the strap which are stretchable are persuading towards contraction meaning they are applying a lifting force from the upper connection, 34, to the guide and from the guide to the foot, 23. As the person ambulates in the direction of travel they lift their hips, preferably with the aid of a cane, crutch or walker, and the persuasion of the strap provides a lifting portion which draws the knee upward towards a near normal gait position. Simultaneously, the lower portion of the strap persuades the foot upward to a position which is approximately perpendicular to the lower leg. The degree of persuasion of the upper portion is preferably equal to the weight of the leg and the degree of persuasion of the lower portion is preferably equal to the downward weight of the foot each of which can be determined analytically or by gait analysis. If the leg does not rotate at the hip and bend at the knee upon lifting the hip the persuasion of the upper portion is insufficient and if the leg can not be straightened by the user once the foot reaches the ground and the person lifts the hip the persuasion is to strong. Similarly, the persuasion of the lower portion can be determined by the angle of the foot to the ground. If the toe is pointing downward, relative to a normal position, when the foot is elevated the persuasion is to low and if the toe is point upward, relative to a normal position, the persuasion is too high.

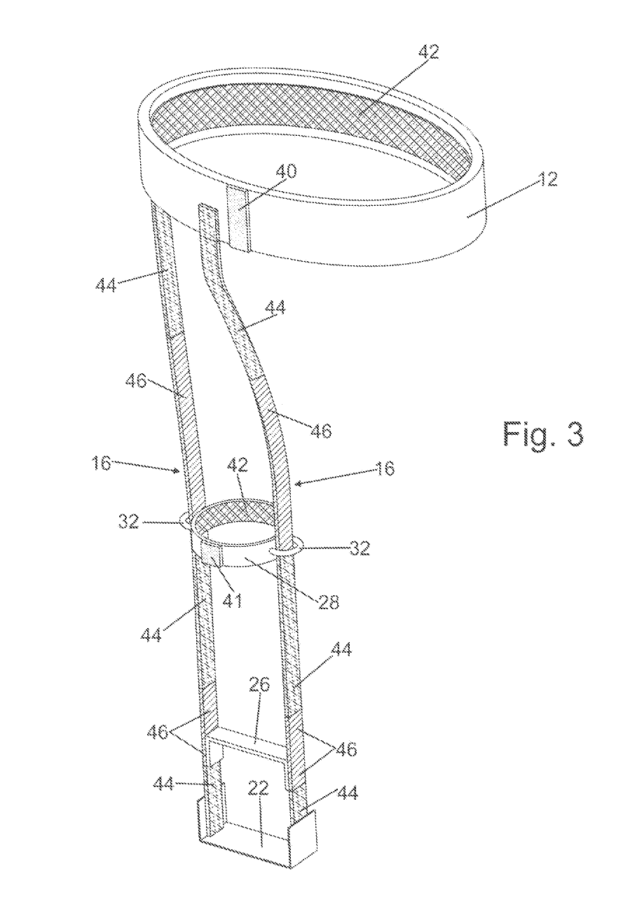

An embodiment of the invention will be described with reference to FIG. 3 wherein a leg lift walking aid is illustrated in isolated view. In FIG. 3, the waist band, 12, comprises an optional but preferable waist band latch, 40, which allows the waist band to be wrapped around the waist and latched thereon with the appropriate tautness. The central band, 28, comprises an optional but preferable central band latch, 41, which may be the same type latch as, or a different type latch than, the waist band latch. It is preferable that the waist band latch and the central band latch be adjustable such that the circumference of the respective device can be adjusted to account for different size wearers and weight fluctuations. While not limited thereto the latch may include snaps, buttons, buckles, adhesives, hook and loop fasteners and the like. Hook and loop fasteners, such as Velcro®, are particularly preferred due to their widespread availability, ease of use, ease of adjustment, excellent adhesion capabilities and relative light weight.

With continued reference to FIG. 3, at least one of the waist band or central band has on the interior thereof an anti-slip material, 42, as described elsewhere herein. The anti-slip material can be integral to the waist band or central band or attached thereto.

With continued reference to FIG. 3, the guides, 32, preferably allow the strap to slide therein. In one embodiment the strap is reversibly attached to the guide thereby isolating stretching of the upper strap, above the guide, from that of the lower strap, below the guide. The transition, 26, may bring the two straps into close proximity, as in FIG. 1, or the transition may form a separation between the two straps. The foot plate, 22, is not particularly limited with the proviso that the foot plate remains in position on the bottom of the shoe or foot without sliding towards or away from the toes. It is preferable to have a non-slip surface on at least the bottom of the foot plate to insure the foot plate does not slide on the walking surface. In a preferred embodiment the foot plate is on the outside of the shoe and wraps at least partially around the shoe. In one embodiment the foot plate is inside the shoe. In one embodiment the foot plate is integral to the shoe.

With continued reference to FIG. 3, the straps comprise at least one portion which does not readily stretch during use, referred to herein as a non-stretch strap, 44, and at least a portion which does stretch during use, referred to herein as stretch strap, 46. Though not limited thereto, non-stretch strap is preferable at the junction of the waist band and strap and at the junction of the foot plate and strap. It is preferable that each stretch strap portion comprise a multiplicity of bands since this allows for easy addition, or subtraction of the degree of persuasion. As bands are added the degree of persuasion of the stretch strap increases. Neoprene rubber straps with a diameter of about 0.5 mm to 3 mm provide sufficient persuasion with 1 to 10 bands preferably used. More preferably, a neoprene rubber strap with a diameter of 1 mm to 2 mm is suitable. It is preferable to include a gathered cloth sleeve over, at least, the stretchable strap for aesthetics and comfort.

The invention has been described with reference to the preferred embodiments without limit thereto. One of skill in the art would realize additional embodiments and improvements which are not specifically set forth herein but which are within the scope of the invention as more specifically set forth in the claims appended hereto.

1. A leg lift walking aid comprising:
a waist band suitable for wearing around a user;
a foot plate capable of being placed under a foot of said user;
a pair of straps between said waist band and said foot plate wherein each strap of said straps comprises at least one flexible portion and at least one non-flexible portion; and
a central band suitable for wearing on a leg, below a knee, of a user wherein said central band comprises at least one guide wherein said guide receives at least one said strap.

2. The leg lift walking aid of claim 1 wherein at least one of said waste band or said central band further comprises a latch.

3. The leg lift walking aid of claim 1 wherein said latch is selected from the group consisting of a snap fastener, a button fastener, a buckle fastener, an adhesive fastener and a hook and loop fastener.

4. The leg lift walking aid of claim 1 wherein at least one of said waste band or said central band further comprises an inner lining.

5. The leg lift walking aid of claim 4 wherein said inner lining comprises a rubber coated fiber or rubber coated fabric.

6. The leg lift walking aid of claim 4 wherein said inner lining comprises a screen pattern.

7. The leg lift walking aid of claim 1 wherein said waste band has a width of at least 7.62 cm to no more than 30.4 cm.

8. The leg lift walking aid of claim 1 wherein said central band has a width of at least 7.62 cm to no more than 15.24 cm.

9. The leg lift walking aid of claim 1 wherein said foot plate is under said shoe, in said shoe or integral to said shoe.

10. The leg lift walking aid of claim 1 wherein said strap comprises multiple stretchable portions.

11. A method of walking with a first leg of limited functionality comprising:
wearing a leg lift walking aid wherein said leg lift walking leg comprises:
a waist band suitable for wearing around a user above a hip joint;
a foot plate capable of being placed under a first foot of said first leg of said user;
a pair of straps between said waist band and said foot plate wherein each strap of said straps comprises at least one flexible portion and at least one non-flexible portion; and
a central band suitable for wearing on said first leg below a knee of said first leg of said user wherein said central band comprises a pair of guides wherein each guide of said guides receives one said strap;
standing upright with said first foot and a second foot on a surface;
lifting hips away from said surface wherein said first leg is persuaded to rotate at said hip joint and bend at said knee;
ambulating forward; and
returning said first foot to said surface.

12. The method of walking with a first leg of limited functionality of claim 11 wherein at least one of said waste band or said central band further comprises a latch.

13. The method of walking with a first leg of limited functionality of claim 11 wherein said latch is selected from the group consisting of a snap fastener, a button fastener, a buckle fastener, an adhesive fastener and a hook and loop fastener.

14. The method of walking with a first leg of limited functionality of claim 11 wherein at least one of said waste band or said central band further comprises an inner lining.

15. The method of walking with a first leg of limited functionality of claim 14 wherein said inner lining comprises a rubber coated fiber or rubber coated fabric.

16. The method of walking with a first leg of limited functionality of claim 14 wherein said inner lining comprises a screen pattern.

17. The method of walking with a first leg of limited functionality of claim 11 wherein said waste band has a width of at least 7.62 cm to no more than 30.4 cm.

18. The method of walking with a first leg of limited functionality of claim 11 wherein said central band has a width of at least 7.62 cm to no more than 15.24 cm.

19. The method of walking with a first leg of limited functionality of claim 11 wherein said foot plate is under said shoe, in said shoe or integral to said shoe.

20. The method of walking with a first leg of limited functionality of claim 11 wherein said strap comprises multiple stretchable portions.
